(19) United States
(12) Patent Application Publication
(10) Pub. No.: US 2010/0301012 A1
(43) Pub. Date: Dec. 2, 2010

(54) DEVICE AND METHOD FOR PRODUCING MICROWAVE PLASMA WITH A HIGH PLASMA DENSITY

(75) Inventor: Ralf Spitzl, Troisdorf (DE)

Correspondence Address: D. PETER HOCHBERG CO. L.P.A., 1940 EAST 6TH STREET, CLEVELAND, OH, 44114, US

(73) Assignee: Iplas Innovative Plasma Systems GmbH, Troisdorf (DE)

(21) Appl. No.: 12/311,810

(22) PCT Filed: Oct. 11, 2007
(86) PCT No.: PCT/EP2007/008839

§ 371 Date: Jul. 20, 2009

(30) Foreign Application Priority Data
Oct. 16, 2006 (DE) 10 2006 048 814.8

(51) Int. Cl. H05H 1/46; B01J 19/08; A61L 2/00; C23C 16/511; C23F 1/00; B08B 6/00

(52) U.S. Cl. 216/69; 422/186; 422/21; 422/186.05; 118/723 MW; 156/345.41; 118/723 AN; 134/1.1; 427/575

(57) ABSTRACT

A device for producing microwave plasma with a high plasma density. The device comprises at least one microwave supply that is surrounded by an outer dielectric tube. The microwave supply is surrounded by, in addition to the outer dielectric tube, at least one inner dielectric tube that extends inside the outer dielectric tube. The outer dielectric tube and the at least one inner dielectric tube form at least one area that is suitable for receiving and conducting a fluid. The device can be cooled by a fluid. A process gas can be fed into the plasma region by the outer dielectric tube.

### CROSS-REFERENCE TO RELATED APPLICATIONS

This application is a National Stage application of International Application No. PCT/EP2007/008839, filed on Oct. 11, 2007, which claims priority of German application number 10 2006 048 814.8, filed on Oct. 16, 2006, both of which are incorporated herein by reference in their entireties.

### BACKGROUND OF THE INVENTION

### Field of the Invention

The present invention relates to a device for producing microwave plasmas with a high plasma density, comprising at least one microwave feed that is surrounded by at least one dielectric tube. Furthermore, the present invention relates to a method for producing microwave plasmas with a high plasma density by using the device.

### DESCRIPTION OF THE PRIOR ART

Devices for generating microwave plasmas are being used in the plasma treatment of workpieces and gases. Plasma treatment is used, for example, for coating, cleaning, modifying and etching workpieces, for treating medical implants, for treating textiles, for sterilisation, for light generation, preferably in the infrared to ultraviolet spectral range, for converting gases or for gas synthesis, as well as in waste gas purification technology. To this end, the workpiece or gas to be treated is brought into contact with the plasma or the microwave radiation.

The geometry of the workpieces to be treated ranges from flat substrates, fibres and webs, to any configuration of shaped articles.

The most important process gases are inert gases, fluorine-containing and chlorine-containing gases, hydrocarbons, furans, dioxins, hydrogen sulfides, oxygen, hydrogen, nitrogen, tetrafluoromethane, sulfur hexafluoride, air, water, and mixtures thereof In the purification of waste gases by means of microwave-induced plasma, the process gas consists of all kinds of waste gases, especially carbon monoxide, hydrocarbons, nitrogen oxides, aldehydes and sulfur oxides. However, these gases can be used as process gases for other applications as well.

Devices that generate microwave plasmas have been described in the documents WO 98/59359 A1, DE 198 480 22 A1 and DE 195 032 05 C1.

The above-listed documents have in common that they describe a microwave antenna in the interior of a dielectric tube. If microwaves are generated in the interior of such a tube, surface waves will form along the external side of that tube. In a process gas which is under low pressure, these surface waves produce a linear elongate plasma. Typical low pressures are 0.1 mbar-10 mbar. The volume in the interior of the dielectric tube is typically under ambient pressure (generally normal pressure; approx. 1013 mbar). In some embodiments a cooling gas flow passing through the tube is used to cool the dielectric tube.

To feed the microwaves, hollow waveguides and coaxial conductors are used, inter alia, while antennas and slots, among others, are used as the coupling points in the wall of the plasma chamber. Feeds of this kind for microwaves and coupling points are described, for example, in DE 423 59 14 and WO 98/59359 A1.

The microwave frequencies employed for generating the plasma are preferably in the range from 800 MHz to 2.5 GHz, more preferably in the range from 800 MHz to 950 MHz and 2.0-2.5 GHz, but the microwave frequency may lie in the entire range from 10 MHz up to several 100 GHz.

DE 198 480 22 A1 and DE 195 032 05 C1 describe devices for the production of plasma in a vacuum chamber by means of electromagnetic alternating fields, comprising a conductor that extends, within a tube of insulating material, into the vacuum chamber, with the insulating tube being held at both ends by walls of the vacuum chamber and being sealed with respect to the walls at its outer surface. The ends of the conductor are connected to a generator for generating the electromagnetic alternating fields.

A device for producing homogenous microwave plasmas according to WO 98/59359 A1 enables the generation of particularly homogeneous plasmas of great length, even at higher process pressures, as a result of the homogeneous input coupling.

The possible applications of the above-mentioned plasma sources are limited by the high energy release of the plasma to the dielectric tube. This energy release may result in an excessive heating of the tube and ultimately lead to the destruction thereof. For that reason, these sources are typically operated at microwave powers of about 1-2 kW at a correspondingly low pressure (approximately 0.1-0.5 mbar). The process pressures can also be 1 mbar-100 mbar, but only under certain conditions and at a correspondingly low power, in order not to destroy the tube.

With the above-mentioned devices, typical plasma lengths of 0.5-1.5 m can be achieved. With plasmas of almost 100% argon it is possible to achieve greater lengths, but such plasmas are of little technical importance.

Another problem with such plasma sources lies in the channelling of the process gas, especially at higher process gas pressures (above 1 mbar). The reason for this is that with increasing radial distance from the dielectric tube, the plasma density decreases strongly. This makes it more difficult to supply new process gas to the areas of high charge carrier density. In addition, at higher process pressures, the thermal power dissipated to the dielectric tube increases.

However, higher process gases are preferred since they frequently result in a clear, tenfold to hundredfold, increase in the process velocity.

### SUMMARY OF THE PRESENT INVENTION

It is the object of the present invention to overcome the above-mentioned disadvantages and thereby to achieve an increase in plasma concentration and in the process gas pressure.

In accordance with the invention, this object is achieved by a device for generating microwave plasmas in accordance with the present invention. This device comprises at least one microwave feed that is surrounded by an inner dielectric tube. Said inner dielectric tube is in turn surrounded by at least one outer dielectric tube. A space is thereby formed which is suitable for receiving and conducting a fluid.

By means of the device it is possible to conduct a fluid through the above-described double-tube arrangement in an advantageous manner, which fluid can be used for cooling or for being supplied to the process gas.

Suitable microwave feeds are known to those skilled in the art. Generally, a microwave feed consists of a structure which is able to emit microwaves into the environment. Structures that emit microwaves are known to those skilled in the art and can be realised by means of all known microwave antennae and resonators comprising coupling points for coupling the microwave radiation into a space. For the above-described device, cavity resonators, bar antennas, slot antennas, helix antennas and omnidirectional antennas are preferred. Coaxial resonators are especially preferred.

In service, the microwave feed is connected via microwave feed lines (hollow waveguides or coaxial conductors) to a microwave generator (e.g. klystron or magnetron). To control the properties of the microwaves and to protect the elements, it is furthermore possible to introduce circulators, insulators, tuning elements (e.g. 3-pin tuners or E/H tuners) as well as mode converters (e.g. rectangular and coaxial conductors) in the microwave supply.

The dielectric tubes are preferably elongate. This means that the tube diameter: tube length ratio is between 1:1 and 1:1000, and preferably 1:10 to 1:100. The two tubes may be equally long or be of a different length. Furthermore, the tubes are preferably straight, but they may also be of a curved shape or have angles along their longitudinal axis.

The cross-sectional surface of the tubes is preferably circular, but generally any desired surface shapes are possible. Examples of other surface shapes are ellipses and polygons.

The elongate shape of the tubes produces an elongate plasma. An advantage of elongate plasmas is that by moving the plasma device relative to a flat workpiece it is possible to treat large surfaces within a short time.

The dielectric tubes should, at the given microwave frequency, have a low dielectric loss factor tan δ for the microwave wavelength used. Low dielectric loss factors tan δ are in the range from 10⁻² to 10⁻⁷.

Suitable dielectric materials for the dielectric tubes are metal oxides, semimetal oxides, ceramics, plastics, and composite materials of these substances. Particularly preferred are dielectric tubes made of silica glass or aluminium oxide with dielectric loss factors tan δ in the range from 10⁻³ to 10⁻⁴. The dielectric tubes here may be made of the same material or of different materials.

According to one particular embodiment, the dielectric tubes are closed at their end faces by walls.

A gas-tight or vacuum-tight connection between the tubes and the walls is advantageous. Connections between two workpieces are known to those skilled in the art and may, for example, be glued, welded, clamped or screwed connections. The tightness of the connection may range from gas-tight to vacuum-tight, with vacuum-tight meaning, depending on the working environment, tightness in a rough vacuum (300-1 hPa), fine vacuum (1-10⁻³ hPa), high vacuum (10⁻³-10⁻⁷ hPa) or ultrahigh vacuum (10⁻⁷-10⁻¹² hPa). Generally, the term “vacuum-tight” here refers to tightness in a rough or fine vacuum.

The walls may be provided with passages, through which a fluid can be conducted. The size and shape of the passages can be chosen at will. Depending on the application, each wall may contain at least one passage. In a preferred embodiment, there are no passages in the region that is covered by the face end of the inner dielectric tubes.

The fluid is conducted through the space between the outer dielectric tube and the inner dielectric tube, and is fed and discharged, respectively, via the apertures in the walls at the face ends of the dielectric tubes.

The flow velocity and the flow behaviour (laminar or turbulent) of the dielectric fluid flowing through the dielectric tube is to be chosen such that the fluid, in particular if it is a liquid, has good contact with the boundary of the dielectric tube and that, in addition, where a liquid fluid is used, there does not occur any evaporation of the dielectric liquid. How the flow velocity and flow behaviour can be controlled by means of pressure and by means of the shape and size of the passages is known to those skilled in the art.

Suitable for use as the dielectric fluid are both a gas and a dielectric fluid.

However, cooling of the dielectric tube by means of a fluid cannot be realised in an easy fashion since the energy input of the microwaves to the fluid leads to the heating of the latter.

Any additional heating of the fluid will decrease the cooling effect on the dielectric tube. This decrease in the cooling performance can also, at a high microwave absorption by the fluid, lead to a negative cooling performance. This corresponds to an additional heating of the dielectric tube.

To keep the heating of the fluid by the microwaves as low as possible, the fluid must, at the wavelength of the microwaves, have a low dielectric loss factor tan δ in the range of 10⁻² to 10⁻⁷. This prevents a microwave power input into the fluid or reduces said input to an acceptable degree.

Because of their higher thermal coefficient, liquid fluids absorb more thermal power than gaseous fluids.

An example of such a dielectric liquid is an insulating oil that has a low dielectric loss factor. Insulating oils are, for instance, mineral oils, olefins (e.g. poly-alpha-olefin) or silicone oils (e.g. COOLANOL® or dimethylpolysiloxane). Hexadimethylsiloxane is preferred as the dielectric liquid.

By means of this fluid cooling of the outer dielectric tube, it is possible to reduce the heating of the outer dielectric tube. This enables higher microwave powers which, in turn, lead to an increase in the concentration of the plasma at the outside of the outer dielectric tube. In addition, the cooling enables a higher process pressure than in uncooled plasma generators.

By contrast to the gas cooling according to DE 195 032 05, where the cooling gas is in contact with the microwave feed, in the device described herein the contact between the fluid and the microwave feed is prevented by the double-tube arrangement, thereby excluding any possibility of the fluid reacting with the microwave feed. Furthermore, this separation of fluid and microwave feed greatly facilitates the maintenance of the microwave feed.

In a preferred embodiment according to the invention, the material of the outer dielectric tube is replaced by a porous dielectric material. Suitable porous dielectric materials are ceramics or sintered dielectrics, preferably aluminium oxide. However, it also possible to provide tube walls made of silica glass or metal oxides that have small holes.

When a gas flows between the dielectric tubes, part of the gas escapes through said pores. Since the highest microwave field strengths are present at the surface of the outer dielectric tube, the gas molecules, upon passing through the outer dielectric tube, travel through the zone of the highest ion density.

Furthermore, after passing through the pores, the gas has a resultant movement direction radially away from the tube.

If the same gas is used for cooling as is used as the process gas, the portion of the excited particles is increased by the passage of the process gas through the region of the highest microwave intensity. In this way, an efficient transport of excited particles to the workpiece is ensured. This increases both the concentration and the flow of the excited particles.

Furthermore, such an arrangement is also particularly suitable for carrying out pure gas conversion processes such as waste gas purification or gas synthesis processes. Further process gases can, if required, be fed through further porous tubes of the processing chamber.

Due to the porosity of the outer dielectric tube and the gas pressure, the flow (molecules per area per time) of the process gas or process gas mixture is governed by the outer dielectric tube.

Furthermore, in this waste gas purification method, all gas molecules have to pass through the tube wall, and thus through the region of highest ion density. This constitutes an advantage over established methods, wherein the furnace chamber is located in the interior of a volume and the microwaves are irradiated from outside. With an established method, the portion of the purified waste gas is smaller than in the method presented herein since in such a conventional method those portions of the gas which are in the vicinity of the volume are not ionised due to the low field strengths which are present there.

Any known gas may be used as the process gas. The most important process gases are inert gases, fluorine-containing and chlorine-containing gases, hydrocarbons, furans, dioxins, hydrogen sulfides, oxygen, hydrogen, nitrogen, tetrafluoromethane, sulfur hexafluoride, air, water, and mixtures thereof. In the purification of waste gases by means of microwave-induced plasmas, the process gas consists of all kinds of waste gases, especially carbon monoxide, hydrocarbons, nitrogen oxides, aldehydes and sulfur oxides. However, these gases can be used as process gases for other applications as well.

According to a further embodiment, a further dielectric tube may be installed within the outer dielectric tube, said further dielectric tube surrounding the inner dielectric tube and likewise being connected with the walls at its end faces in a gas-tight or vacuum-tight manner. In this embodiment, the space between the outer dielectric tube and the inner dielectric tube is divided into an outer and an inner space.

If the process gas is guided through the outer space and a fluid is guided through the inner space, it is possible to cool the inner dielectric tube and the microwave structure. This, in turn, enables a better process performance. The fluid should not absorb the microwaves. Especially where a liquid is used as the fluid, the liquid should have a low dielectric loss factor tan δ in the range of 10⁻² to 10⁻⁷ for the microwave wavelength used.

In order to further reduce the microwave power requirement for the above-mentioned plasma sources, according to another preferred embodiment it is possible for a metallic jacket to be applied around the outer dielectric tube, said jacket partially covering the tube. This metallic jacket here acts as a microwave shield and may be made, for example, of a metallic tube, a bent sheet metal, a metal foil, or even a metallic layer, and may be plugged or electroplated thereon, or applied thereon in another way. Such metallic microwave shields are able to limit the angular range in which the generation of the plasma takes place as desired (e.g. 90° , 180° or 270°) and thereby reduce the power requirement accordingly.

Especially in the case of the embodiment comprising a metallic jacket of the devices for generating microwave plasmas, it is possible to treat broad material webs with a plasma at a low power loss. The jacket shields that region of the space present in the device which does not face the workpiece, and there is generated only a narrow plasma strip between the workpiece and the device, over the entire width of the workpiece.

All of the above-described devices for plasma generation, during operation, form a plasma at the outside of the dielectric tube. In a normal case, the device will be operated in the interior of a chamber (plasma chamber). This plasma chamber may have various shapes and apertures and serve various functions, depending on the operating mode. For example, the plasma chamber may contain the workpiece to be processed and the process gas (direct plasma process), or process gases and openings for plasma discharge (remote plasma process, waste gas purification).

In one method for producing microwave plasmas in an above-described device, a fluid is guided through the space between the inner dielectric tube and the outer dielectric tube, preferably through passages provided in the walls. In this case, the fluid may be a gas or a liquid.

The pressure of the fluid may be above, below or equal to the atmospheric pressure.

In an advantageous embodiment, a gaseous fluid, preferably a process gas, more preferably a waste gas, is conducted through the porous tube of the above-described device, comprising a porous external tube, and is thereby fed to the plasma process. The fluid here preferably has a low dielectric loss factor tan δ in the range of 10⁻² to 10⁻⁷.

According to another advantageous embodiment, in the above-described device, comprising an inner middle and an outer dielectric tube, a gas, preferably a process gas, flows in the space between the outer dielectric tube and the middle dielectric tube, and a fluid flows in the space between the inner dielectric tube and the middle dielectric tube, said fluid preferably having a low dielectric loss factor tan δ. The outer dielectric tube here preferably has a porous wall.

### BRIEF DESCRIPTION OF THE DRAWINGS

In the following, the invention will be explained, by way of example, by means of the embodiments which are schematically represented in the drawings.

FIG. 1 shows a cross-sectional drawing and a longitudinal-sectional drawing of the device according to the present invention.

FIG. 2 shows a cross-sectional drawing and a longitudinal-sectional drawing of the device according to the present invention, comprising a porous outer dielectric tube.

FIG. 3 shows a cross-sectional drawing and a longitudinal-sectional drawing of the device according to the present invention, comprising an additional cooling.

FIG. 4 shows a cross-sectional drawing of an embodiment of the present invention comprising a metal jacket.

FIG. 5 shows a longitudinal- sectional drawing of the device according to the present invention, the device being installed in a plasma chamber.

FIG. 6A shows a perspective view of an embodiment of the present invention for treating large-area workpieces.

FIG. 6B shows a cross-sectional view of the embodiment of the present invention for treating large-area workpieces as shown in FIG. 6A.

### DETAILED DESCRIPTION OF THE PRESENT INVENTION

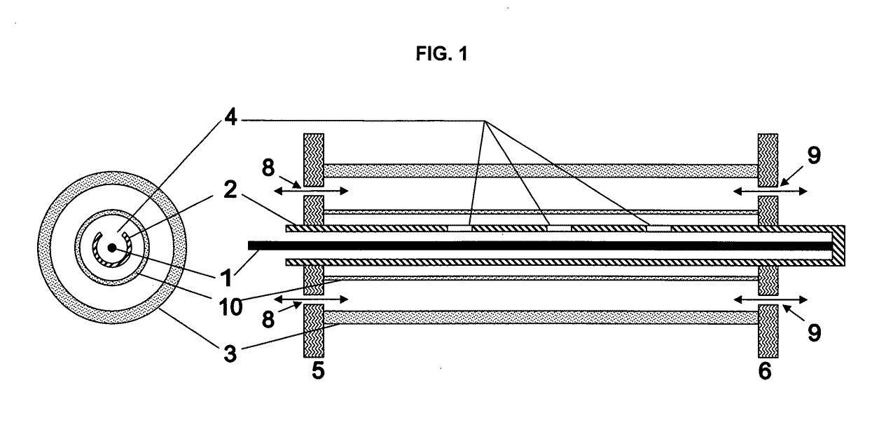

FIG. 1 shows a cross-section and a longitudinal section of a device for generating microwave plasmas, comprising a microwave feed that is configured in the form of a coaxial resonator. Said microwave feed contains an inner conductor (1), an outer conductor (2) and coupling points (4). The microwave feed is surrounded by an outer dielectric tube (3) which separates the microwave feeding region from the plasma chamber (not shown) and on whose outer side the plasma is formed. The outer dielectric tube (3) is connected with the walls (5, 6) in a gas-tight or vacuum-tight manner. Between the coaxial generator and the outer dielectric tube there is inserted an inner dielectric tube (10) that is likewise connected with the walls (5, 6) in a gas-tight or vacuum-tight manner and which, together with the outer dielectric tube (3), forms a space through which a fluid may flow. Said fluid may be fed or discharged, respectively, via the openings (8) and (9).

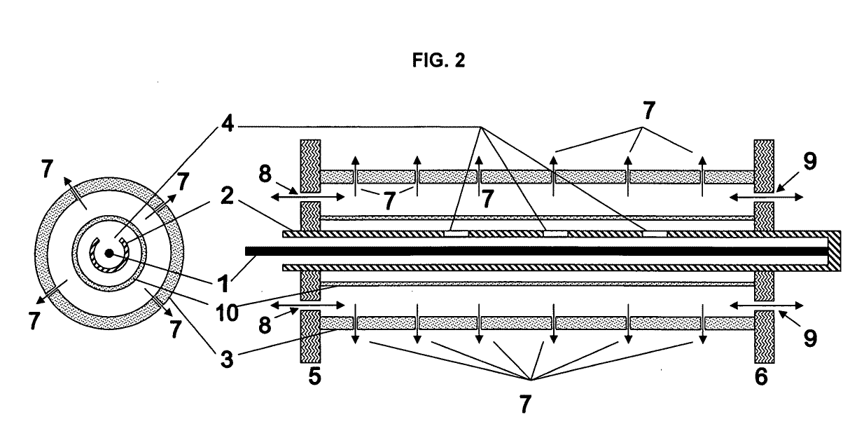

FIG. 2 shows a cross-section and a longitudinal section of an embodiment of the device for generating microwave plasmas as outlined in FIG. 1, wherein the wall of the outer dielectric tube (3) has pores (7). These pores (7) are drawn on a much larger scale for enhanced representation. Via these pores (7), gas can be guided through the outer dielectric tube into the plasma chamber. In the process, it passes through the tube wall of the outer dielectric tube (3), where the field strength of the microwaves, and hence the ionisation of the plasma, is highest.

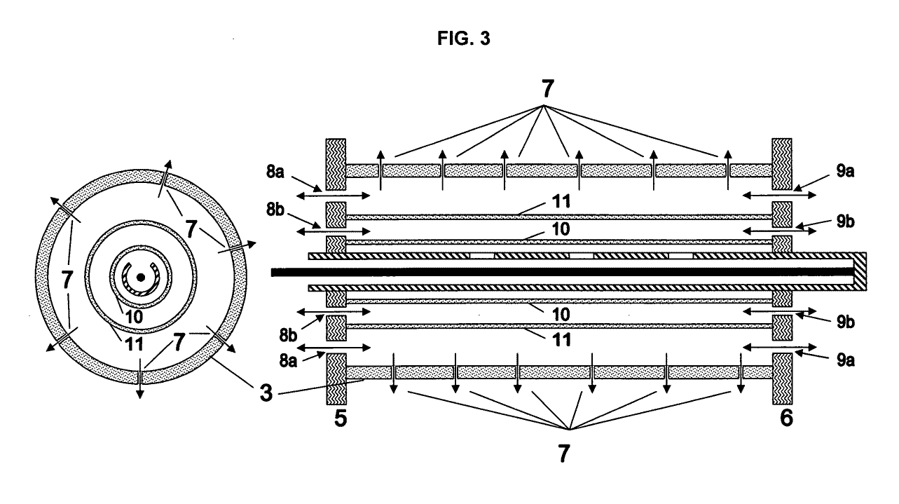

FIG. 3 shows a cross-section and longitudinal section of an embodiment of the device for the generation of microwave plasmas as outlined in FIG. 1, wherein the microwave feed is surrounded by three concentric tubes. This triple-tube arrangement comprises an inner dielectric tube (10) that is surrounded by a middle dielectric tube (11), which, in turn, is surrounded by the outer dielectric tube (3). All three dielectric tubes are connected with the walls (5, 6) in a gas-tight or vacuum-tight manner. A process gas can be fed and discharged, respectively, via the openings (8a) and (9a), and exit through pores (7) in the outer dielectric tube (3). In this Figure, too, the pores (7) are drawn on a much larger scale to enhance the representation. A fluid for cooling the arrangement flows through the inner space between the middle dielectric tube (11) and the inner dielectric tube (10), and can be fed and discharged, respectively, via the openings (8b) and (9b).

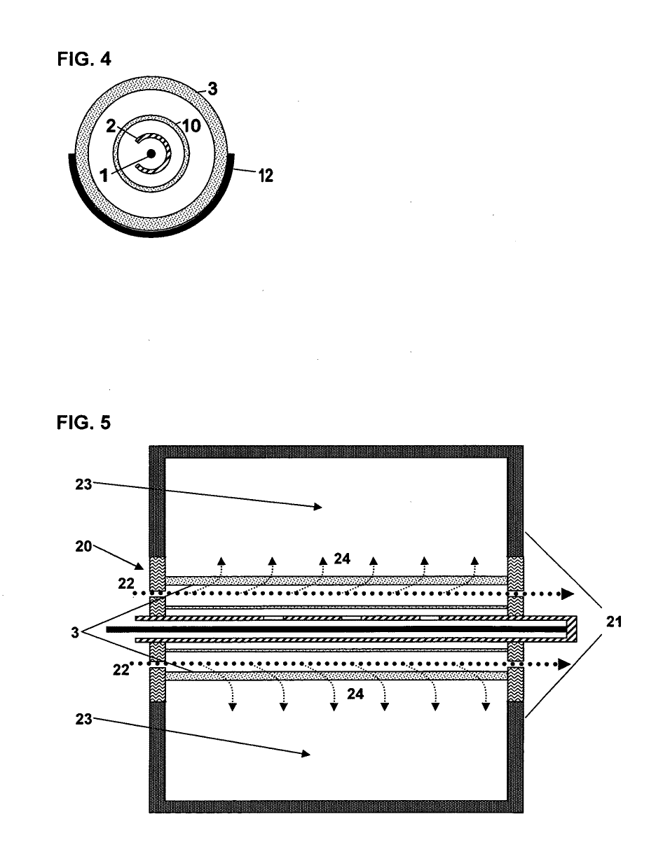

FIG. 4 shows a cross-section of an embodiment of the device shown in FIG. 1, wherein the outer dielectric tube (3) is surrounded by a metallic jacket (12). In the case depicted in FIG. 4, the angular range, which is where the plasma is produced, is limited to 180° by the metallic jacket.

FIG. 5 shows a longitudinal section of a device (20), as described in FIG. 1, which has been installed in a plasma chamber (21). The cooling liquid (22) in this example flows through passages in the two end faces. In service, plasma is formed in the space (23) between the outer dielectric tube (3) and the wall of the plasma chamber.

In a preferred embodiment, wherein the outer dielectric tube (3) has a porous tube wall, as outlined in FIG. 2, the cooling gas, which at the same time serves as the process gas, flows through the tube wall, as indicated by the arrows 24, into the space (23) and forms a plasma.

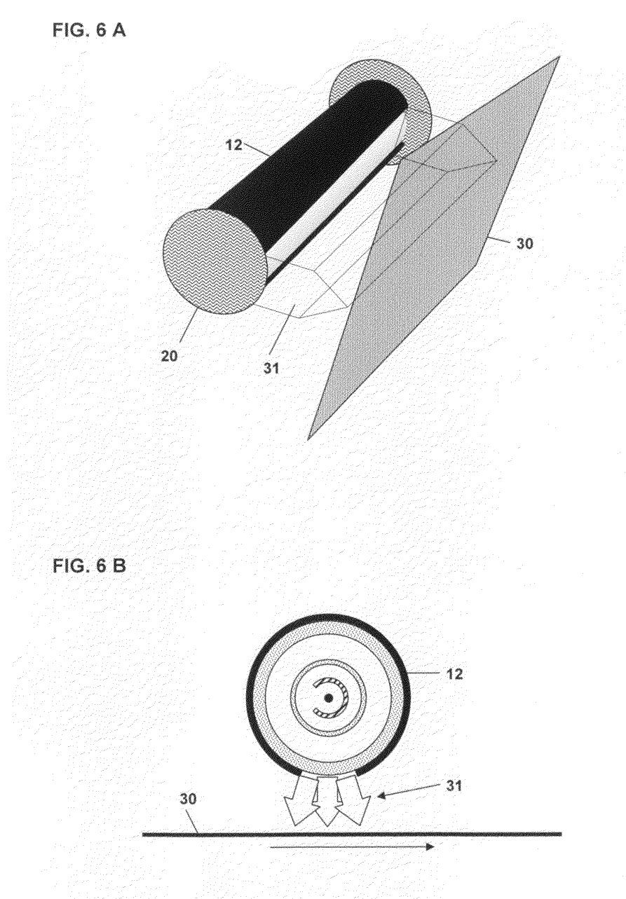

FIGS. 6A and 6B show, in a perspective representation and in a cross-section, an embodiment (20) wherein the major part of the lateral surface of the outer dielectric tube is enclosed by a metal jacket (12) and wherein a plasma (31), which is depicted in the drawing by transparent arrows, can only be formed in a narrow region. In this region, a workpiece (30), moving relative to the device, can be treated with the plasma over a large surface area.

All of the embodiments are fed by a microwave supply, not shown in the drawings, consisting of a microwave generator and, optionally, additional elements. These elements may comprise, for example, circulators, insulators, tuning elements (e.g. three-pin tuner or E/H tuner) as well as mode converters (e.g. rectangular or coaxial conductors).

There are numerous fields of application for the above described device and the above described method. Plasma treatment is employed, for example, for coating, purification, modification and etching of workpieces, for the treatment of medical implants, for the treatment of textiles, for sterilisation, for light generation, preferably in the infrared to ultraviolet spectral region, for conversion of gases or for the synthesis of gases, as well as in gas purification technology. The workpiece or gas to be treated is brought into contact with the plasma or microwave radiation. The geometry of the workpieces to be treated ranges from flat substrates, fibres and webs to shaped articles of any shape.

Due to the increased plasma density and the increased plasma power, it is possible to achieve higher process velocities than with devices and methods according to the prior art.

What has been described above are preferred aspects of the present invention. It is of course not possible to describe every conceivable combination of components or methodologies for purposes of describing the present invention, but one of ordinary skill in the art will recognize that many further combinations and permutations of the present invention are possible. Accordingly, the present invention is intended to embrace all such alterations, combinations, modifications, and variations that fall within the spirit and scope of the appended claims.

I claim:

1. A device for generating microwave plasmas, said device comprising at least one microwave feed and an outer dielectric tube for surrounding said at least one microwave feed, at least one inner dielectric tube for surrounding said at least one microwave feed, said at least one inner dielectric tube extending inside said outer dielectric tube, and walls connecting said outer dielectric tube and said at least one inner dielectric tube the respective end faces of said outer dielectric tube and said at least one inner dielectric tube, and wherein each of the walls comprises at least one passage for fluid, and forms a space for receiving and conducting fluid.

2. The device according to claim 1, wherein there are no passages in the respective regions of the respective walls that is covered by the end face of the at least one inner dielectric tube.

3. The device according to claim 2, wherein a fluid is conducted through the space between the at least one inner dielectric tube and the outer dielectric tube, via said at least one passage.

4. The device according to claim 1, wherein the at least one inner dielectric tube and said outer dielectric tube comprise a material selected from the group consisting of metal oxides, semimetal oxides, ceramics, plastics, and composite materials of these substances.

5. The device according to claim 1, having a lateral surface wherein the outer dielectric tube is porous or gas-permeable, at least in a portion of the lateral surface or in the region of the entire lateral surface.

6. The device according to claim 1, further comprising a middle dielectric tube for surrounding the at least one inner dielectric tube, said middle dielectric tube extending inside the outer dielectric tube.

7. The device according to claim 1, further comprising a metal jacket for partially surrounding the outer dielectric tube.

8. The device according to claim 7, wherein the metal jacket of comprises one selected from the group consisting of a metallic tube segment, a metal foil and a metal layer.

9. The device according to claim 7, wherein the metal jacket leaves a region of the lateral surface of the outer dielectric tube free, said region extending over the entire length of the outer dielectric tube.

10. The device according to claim 1, further comprising a process chamber outside the outer dielectric tube.

11. The device according to claim 1, wherein said at least one microwave feed is selected from the group consisting of a microwave antenna and a cavity resonator with coupling points,

12. The device according to claim 1, further comprising microwave feed lines and a microwave generator, said microwave feed lines connecting said at least one microwave feed with said microwave generator.

13. A method for generating microwave plasmas in a device comprising at least one microwave feed that is surrounded by an inner dielectric tube, said inner dielectric tube being surrounded by an outer dielectric tube, wherein a fluid is conducted through a space between the inner dielectric tube and the outer dielectric tube, said method comprising the steps of:
connecting both the inner dielectric tube and the outer dielectric tube at the respective end faces with walls, said walls having passages, and
conducting said fluid through the passages.

14. The method according to claim 13, wherein there are no passages in the region that is covered by the end face of the inner dielectric tube.

15. The method according to claim 13, wherein the fluid is or contains a liquid.

16. The method according to claim 13, wherein the fluid is or contains a gas.

17. The method according to claim 16, wherein the outer dielectric tube of comprises a porous or gas-permeable material, and further comprising the step of feeding the gas which is conducted through the space between the inner dielectric tube and the outer dielectric tube through the outer dielectric tube to a plasma process.

18. The method according to claim 17, further comprising the step of supplying at least one process gas to the plasma process.

19. The method according to claim 17, further comprising the step of supplying at least one waste gas to the plasma process.

20. The method according to claim 13, wherein the gas pressure in the space between the inner dielectric tube and the outer dielectric tube is higher than or equal to atmospheric pressure.

21. The method according to claim 13, wherein the gas pressure in the space between the inner dielectric tube and the outer dielectric tube is lower than atmospheric pressure.

22. The method according to claim 13, wherein a middle dielectric tube surrounds the inner dielectric tube, the middle dielectric tube extending inside the outer dielectric tube, and the method further comprising the steps of passing a gas passes through a space between the outer dielectric tube and the middle dielectric tube, and passing a fluid passes through a space between the inner dielectric tube and the middle dielectric tube.

23. The method according to claim 13, wherein the fluid has a low dielectric loss factor tan δ in the range of from 10⁻² to 10⁻⁷.

24. Use of a device for generating a plasma for coating, cleaning, modifying and etching workpieces, for treating medical implants, for treating textiles, for sterilisation, for light generation, for light generation in the infrared to ultraviolet spectral region, for converting gases or for gas synthesis, as well as in waste gas purification technology, said device comprising at least one microwave feed and an outer dielectric tube for surrounding said at least one microwave feed, at least one inner dielectric tube for surrounding said at least one microwave feed, said at least one inner dielectric tube extending inside said outer dielectric tube, and walls connecting said outer dielectric tube and said at least one inner dielectric tube at the respective end faces of said outer dielectric tube and said at least one inner dielectric tube, and wherein each of the walls comprises at least one passage for fluid, and forms a space for receiving and conducting fluid.

25. Use of a device according to a method for generating a plasma for coating, cleaning, modifying and etching workpieces, for treating medical implants, for treating textiles, for sterilisation, for light generation, for light generation in the infrared to ultraviolet spectral region, for converting gases or for gas synthesis, as well as in waste gas purification technology said device comprising at least one microwave feed that is surrounded by an inner dielectric tube, said inner dielectric tube being surrounded by an outer dielectric tube, wherein a fluid is conducted through a space between the inner dielectric tube and the outer dielectric tube said method comprising the steps of:
connecting both the inner dielectric tube and the outer dielectric tube at the respective end faces with walls, said walls having passages, and
conducting said fluid through the passages.

26. The device according to claim 4, wherein said at least one inner dielectric tube and said outer dielectric tube comprise a material selected from the group consisting of silica glass and aluminium oxide.

27. The device according to claim 11, wherein said at least one microwave feed is a coaxial resonator.

28. The device according to claim 12, wherein said microwave feed lines are selected from the group consisting of hollow waveguides and coaxial conductors, and wherein said microwave generator is selected from the group consisting of a klystron and a magnetron.
